(19) United States
(12) Patent Application Publication
(10) Pub. No.: US 2010/0010433 A1
(43) Pub. Date: Jan. 14, 2010

(54) MEDICAL SYSTEM WHICH CONTROLS DELIVERY OF A DRUG

(75) Inventors: Ross G. Krogh, Long Grove, IL (US); James F. Martin, Lebanon, OH (US); Paul J. Niklewski, Cincinnati, OH (US)

Correspondence Address: THOMPSON HINE L.L.P.;Intellectual Property Group, P.O. BOX 8801, DAYTON, OH, 45401-8801, US

(73) Assignee: Ethicon Endo-Surgery, Inc., Cincinnati, OH (US)

(21) Appl. No.: 12/499,821

(22) Filed: Jul. 9, 2009

Related U.S. Application Data
(60) Provisional application No. 61/196,708, filed on Jul. 10, 2008.

(51) Int. Cl. A61M 5/168; G06F 15/16

(52) U.S. Cl. 604/66; 709/201

(57) ABSTRACT

A first medical system includes a controller, a plurality of predetermined patient monitors, and a predetermined drug-delivering medical effector. The controller is adapted to choose different groups of the patient monitors for different medical procedures involving the medical effector. A second medical system includes a controller which is adapted to identify, by querying, connected ones of a multiplicity of medical effectors and connected ones of a plurality of patient monitors and is adapted to control at least one connected medical effector using at least some of the connected patient monitors for a predetermined medical procedure.

### CROSS REFERENCE TO RELATED APPLICATIONS

This application claims the benefit of U.S. Provisional Application Ser. No. 61/196,708 filed on Jul. 10, 2008, the entire contents of which are hereby incorporated by reference.

### FIELD OF THE INVENTION

The present invention is related generally to medical technology, and more particularly to a medical system which controls delivery of a drug.

### BACKGROUND OF THE INVENTION

Known medical systems include those which control delivery of a drug to a patient. One known example is a stand-alone IV (intravenous) pump programmed by a user to deliver a volume of a drug at a prescribed fixed flow rate and to alarm before the full volume is delivered. Conventional IV pumps include an IV pump which has an AC-rechargeable battery capable of operating the IV pump (without the IV pump being plugged into an AC power source) for a period of several hours and which is sized to fit into a storage compartment of a hiker's backpack.

Another known example of such a medical system is a preconfigured conscious sedation system having a bedside monitoring unit including a pulse oximeter which measures the pulse rate and the SpO₂ (saturation of peripheral oxygen) level of the patient and including a blood pressure module which measures the systolic blood pressure level and the diastolic blood pressure level of the patient. The bedside monitoring unit travels with the patient from the preparation room to the procedure room. Then, the bedside monitoring unit is connected by a cable to a procedure room unit which has a wired display monitor, wherein the pulse rate, the SpO₂ respiration rate, the systolic blood pressure level, and the diastolic blood pressure level (and other predetermined physiological parameters of the patient) measured by the preconfigured bedside monitoring unit are displayed on the display monitor.

The procedure room unit includes a controller which computes a flow rate to deliver a sedation drug intravenously to the patient for a predetermined medical procedure. The controller computes the flow rate based on all of the predetermined physiological parameters of the patient measured by the preconfigured bedside monitoring unit. The controller sends a flow-rate command to an IV pump assembly, wherein the flow-rate command changes with changes in the measured physiological parameters of the patient.

Still, scientists and engineers continue to seek improved medical systems.

### SUMMARY

An expression of a first embodiment of the invention is for a medical system including a medical-system controller. The controller is operatively connectable to a distributed plurality of predetermined patient monitors to receive at least one physiological parameter measured by each of the plurality of patient monitors. The controller is adapted to choose a first group of the patient monitors for a predetermined first medical procedure and a predetermined drug-delivering first medical effector. The controller is operatively connectable to the first medical effector and, when operatively connected, is adapted to control a flow rate of a drug from the first medical effector to a patient for the first medical procedure using at least the received measured physiological parameters from the chosen first group of the patient monitors, when the controller is operatively connected to the patient monitors, without using any other physiological parameter of the patient. The controller is adapted to choose a second group of the patient monitors different from the first group for a different predetermined second medical procedure and the first medical effector. The controller, when operatively connected to the first medical effector, is adapted to control a flow rate of a drug from the first medical effector to the patient for the second medical procedure using at least the received measured physiological parameters from the chosen second group of the patient monitors, when the controller is operatively connected to the patient monitors, without using any other physiological parameter of the patient.

An expression of a second embodiment of the invention is for a medical system including a medical-system controller. The controller is connectable to up to a distributed plurality P of patient monitors which are operatively connectable to a patient to each measure at least one physiological parameter of the patient. The controller is connectable to up to a distributed multiplicity M of drug-delivering medical effectors which are operatively connectable to the patient and which are adapted to identify themselves when queried. The controller is adapted to identify, by querying, connected ones of the M medical effectors and connected ones of the P patient monitors. The controller is adapted to control at least one of the identified connected ones of the M medical effectors using at least the physiological parameters supplied by at least some of the identified connected ones of the P patient monitors for a predetermined medical procedure.

Several benefits and advantages are obtained from one or more of the expressions of the embodiments of the invention. In one example of the first embodiment, a medical-system controller is provided which is connectable to a distributed plurality of predetermined patient monitors and which adapts its choice of patient monitors to use depending on the particular medical procedure to be performed. In one example of the second embodiment, a medical-system controller is provided which is connectable to a distributed plurality of patient monitors and a distributed multiplicity of drug-delivering medical effectors, which identifies connected one of the medical effectors and patient monitors, and which controls at least one of the connected ones of the medical effectors using at least some of the connected ones of the patient monitors for a predetermined medical procedure.

### BRIEF DESCRIPTION OF THE FIGURES

FIG. 1 is schematic diagram of a first embodiment of a medical system of the invention wherein a medical-system controller is connected to three predetermined patient monitors and two predetermined drug-delivering medical effectors;

FIG. 2 is a schematic diagram of a second embodiment of a medical system of the invention including a medical-system controller, five patient monitors and three drug-delivering medical effectors, wherein the medical-system is shown connected to three of the five patient monitors and to two of the three drug-delivering medical effectors;

FIG. 3 is a schematic front view of a third embodiment of a medical system of the invention showing a folding backpack pouch in an unfolded state;

FIG. 4 is a top view of the backpack pouch of FIG. 3 showing a laptop computer, a patient monitoring module, a patient gas-exchange module, a drug-delivery module, and a battery unit stored in the backpack pouch;

FIG. 5 is a view, as in FIG. 4, but with the backpack pouch in a folded state;

FIG. 6 is a diagrammatic view showing the interconnections of the laptop computer and the three modules of FIG. 4;

FIG. 7 is an enlarged view of a portion of FIG. 4 showing the connecting cables affixed to the backpack pouch and having plugs for connecting to the modules and the battery unit; and

FIG. 8 is a schematic view of the battery unit removed from the backpack pouch.

### DETAILED DESCRIPTION

Before explaining the expressions of several embodiments of the invention in detail, it should be noted that each is not limited in its application or use to the details of construction and arrangement of parts, instructions, and steps illustrated in the accompanying drawings and description. The illustrative embodiments of the invention may be implemented or incorporated in other embodiments, variations, and modifications, and may be practiced or carried out in various ways. Furthermore, unless otherwise indicated, the terminology employed herein has been chosen for the purpose of describing the illustrative expressions of the embodiments of the present invention for the convenience of the reader and not for the purpose of limiting the invention.

It is further understood that any one or more of the following-described expressions of a medical system, implementations, etc. can be combined with any one or more of the other following-described expressions of a medical system, implementations, etc.

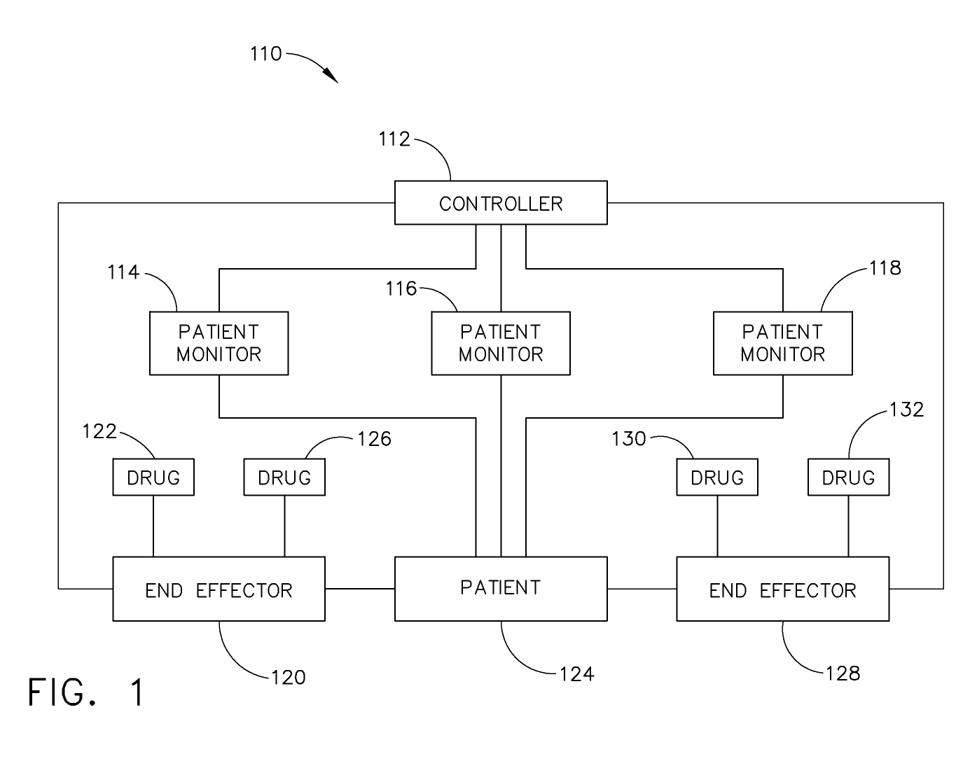

A first embodiment of the invention is shown in FIG. 1. A first expression of the embodiment of FIG. 1 is for a medical system 110 including a medical-system controller 112. The controller 112 is operatively connectable to a distributed plurality of predetermined patient monitors 114, 116, and 118 to receive at least one physiological parameter measured by each of the plurality of patient monitors 114, 116, 118. The controller 112 is adapted to choose a first group of the patient monitors for a predetermined first medical procedure and a predetermined drug-delivering first medical effector 120. The controller 112 is operatively connectable to the first medical effector 120 and, when operatively connected, is adapted to control a flow rate of a drug 122 from the first medical effector to a patient 124 for the first medical procedure using at least the received measured physiological parameters from the chosen first group of the patient monitors, when the controller 112 is operatively connected to the patient monitors 114, 116, and 118, without using any other physiological parameter of the patient 124. The controller 112 is adapted to choose a second group of the patient monitors different from the first group for a different predetermined second medical procedure and the first medical effector 120. The controller 112, when operatively connected to the first medical effector 120, is adapted to control a flow rate of a drug 126 from the first medical effector 120 to the patient 124 for the second medical procedure using at least the received measured physiological parameters from the chosen second group of the patient monitors, when the controller 112 is operatively connected to the patient monitors 114, 116, and 118, without using any other physiological parameter of the patient 124. It is noted that drug 122 and the drug 124 may be the same drug or different drugs. It is noted that the term “drug” includes a combination drug.

Types of drug-delivering medical effectors include IV pump assemblies which deliver a drug intravenously to a patient, gas delivery pump assemblies which deliver a gaseous drug to a patient via an oral and/or nasal cannula, an electrical stimulator which delivers an electric current (considered a drug for the purposes of describing the embodiments of the invention) to a patient to sedate the patient, and a drug patch and drug-patch control unit wherein the drug patch contains a drug and wherein the drug-patch control unit controls the delivery of the drug to the patient by electrophoresis.

In one example of the first expression of the embodiment of FIG. 1, the patient monitors 114, 116, and 118 include an ECG (electrocardiogram) patient monitor 114, a blood pressure patient monitor 116, and a pulse oximeter patient monitor 118 which measure corresponding physiological parameters of the patient 124. In one variation, the first medical effector 120 is an IV (intravenous) pump assembly. In one illustration of a first medical procedure (e.g., a colonoscopy), the corresponding drug 122 (such as Propofol) has a sedative effect, and the chosen first group of patient monitors is the group consisting of the ECG patient monitor 114, the blood pressure patient monitor 116, and the pulse oximeter patient monitor 118. In one illustration of a second medical procedure (e.g., an operation to remove the wisdom teeth), the corresponding drug 126 (such as Meperidine) has an analgesic effect, and the chosen second group of patient monitors is the group consisting of the ECG patient monitor 114 and the blood pressure patient monitor 116. Other illustrations are left to those skilled in the medical arts. In one utilization, a drug to be used by the first medical effector 120 is identified to the controller 112 by a touch screen (not shown) on the controller 112 which displays a list of pre-programmed drugs.

Another example of a patient monitor (not shown) is a CO₂ (carbon dioxide) patient monitor. An additional example is a sedation level patient monitor which queries a patient for a response (e.g., which buzzes a handpiece at various power levels as a request for the patient to push a button) and which measures the response (e.g., the time delay for the patient to push the button for a particular power level which indicates the level of patient sedation). Other examples are left to those skilled in the medical arts.

It is within the ordinary level of skill of the artisan to obtain and have programmed a medical-system controller 112 which is adapted: to receive inputs from various predetermined patient monitors 114, 116, and 118; to choose a particular group of such patient monitors to use with a predetermined drug-delivering first medical effector 120 for a user-chosen one of a plurality of predetermined medical procedures, and to control the drug flow of a predetermined drug from the first medical effector 120 to the patient 124 using the chosen group of patient monitors without using non-chosen patient monitors.

In one implementation of the first expression of the embodiment of FIG. 1, the controller 112 is adapted to control the flow rate for the first medical procedure despite failure of at least one of the patient monitors of the first group of the patient monitors. In one extension of the first expression of the embodiment of FIG. 1, the medical system 110 includes the plurality of the patient monitors 114, 116, and 118 and the first medical effector 120 wherein each is operatively connected to the controller 112.

In a first application of the first expression of the embodiment of FIG. 1, the controller 112 is adapted to choose a third group of the patient monitors for a third medical procedure and a predetermined drug-delivering second medical effector 128. The controller 112 is operatively connectable to the second medical effector 128 and, when operatively connected, is adapted to control a flow rate of a drug 130 from the second medical effector 128 to the patient 124 for the third medical procedure using at least the received measured physiological parameters from the chosen third group of the patient monitors, when the controller 112 is operatively connected to the patient monitors 114, 116, and 118, without using any other physiological parameters of the patient 124. The third group may be the same or different from the first group. The third group may be the same or different from the second group.

In one variation of the first application, the controller 112 is adapted to control the flow rate for the third medical procedure despite failure of at least one of the patient monitors of the third group of the patient monitors. In one extension of the first application, the medical system 110 includes the plurality of the patient monitors 114, 116, and 118, the first medical effector 120, and the second medical effector 128 wherein each is operatively connected to the controller 112.

In a second application of the first expression of the embodiment of FIG. 1, the controller 112 is adapted to choose a fourth group of the patient monitors for the first medical procedure and a predetermined drug-delivering second medical effector 128. The controller 112 is operatively connectable to the second medical effector 128 and, when operatively connected, is adapted to control a flow rate of a drug 132 from the second medical effector 128 to the patient 124 for the first medical procedure using at least the received measured physiological parameters from the chosen fourth group of the patient monitors, when the controller 112 is operatively connected to the patient monitors 114, 116, and 118, without using any other physiological parameter of the patient 124.

It is noted that the drugs 122, 126, 130 and 132 may be the same drug or different drugs. It is also noted that the fourth group may be the same or different from the first group. It is further noted that the fourth group may be the same or different from the second group. In one variation of the second application, the controller 112 is adapted to control the flow rate of the drug 122 of the first medical effector 120 for the first medical procedure despite failure of the second medical effector 128.

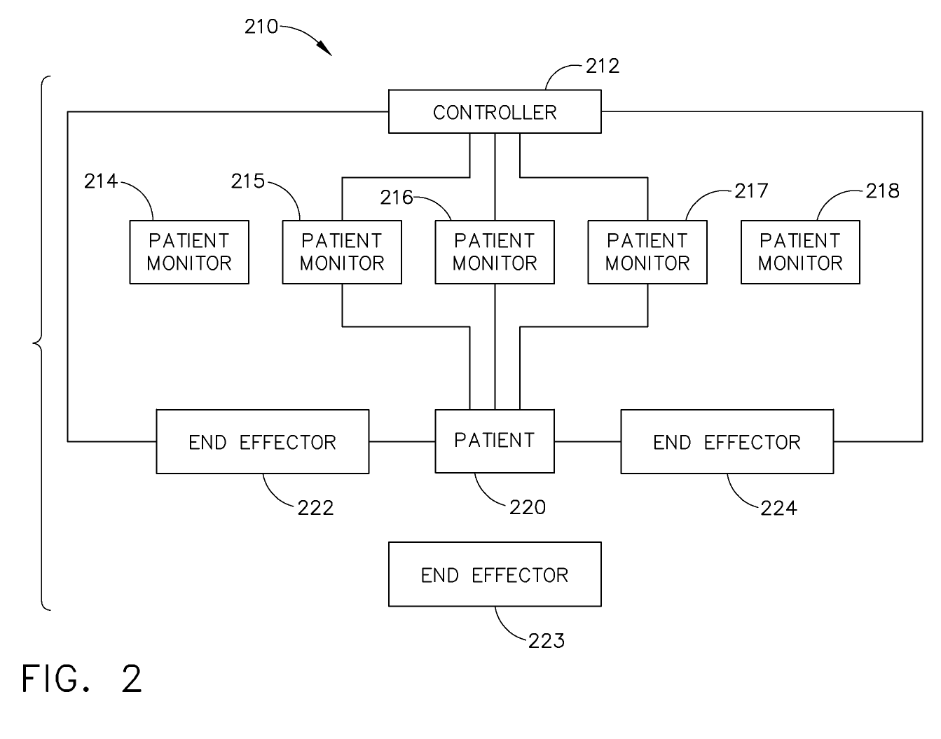

A second embodiment of the invention is shown in FIG. 2. A first expression of the embodiment of FIG. 2 is for a medical system 210 including a medical-system controller 212. The controller 212 is connectable to up to a distributed plurality P of patient monitors 214, 215, 216, 217, and 218 which are operatively connectable to a patient 220 to each measure at least one physiological parameter of the patient 220. The controller 212 is connectable to up to a distributed multiplicity M of drug-delivering medical effectors 222, 223, and 224 which are operatively connectable to the patient 220 and which are adapted to identify themselves when queried. The controller 212 is adapted to identify, by querying, connected ones of the M medical effectors 222-224 and connected ones of the P patient monitors 214-218. The controller 212 is adapted to control at least one of the identified connected ones 222 and 224 of the M medical effectors using at least the physiological parameters supplied by at least some of the identified connected ones 215, 216, and 217 of the P patient monitors for a predetermined medical procedure.

Connections of the patient monitors and the medical effectors may be parallel connections, serial connections, or combinations of both and include wired and wireless connections. It is noted that unconnected ones 214 and 218 of the plurality P of patient monitors and unconnected ones 223 of the multiplicity M of medical effectors my be unconnected in the physical sense for wired connections (as shown in FIG. 2 where only wired connections are present) or unconnected in the operative sense (e.g., a physically connected patient monitor or a wirelessly-connected patient monitor may be turned off and unable to communicate with the controller). It is also noted that P is the number of the plurality of the patient monitors, and M is the number of the multiplicity of medical effectors. In FIG. 2, P equals 5 and M equals 3. Other values of P and M are left to those skilled in the medical arts.

In one extension of the first expression of the embodiment of FIG. 2, the medical system 210 includes the plurality P of the patient monitors 214, 215, 216, 217, and 218 and the multiplicity M of the medical effectors 222, 223 and 224.

In one deployment of the first and/or second embodiment, each peripheral component (i.e. each patient monitor and each medical effector) may have a basic algorithm, wherein the medical system provides more complex behaviors. A peripheral component may serve as a signal repeater increasing the range of wireless peripheral components. A peripheral component may provide its own power through a battery or AC connection or receive power from a wire-connected peripheral component. A peripheral component may provide its own computation and alarm settings or leverage those from another peripheral component.

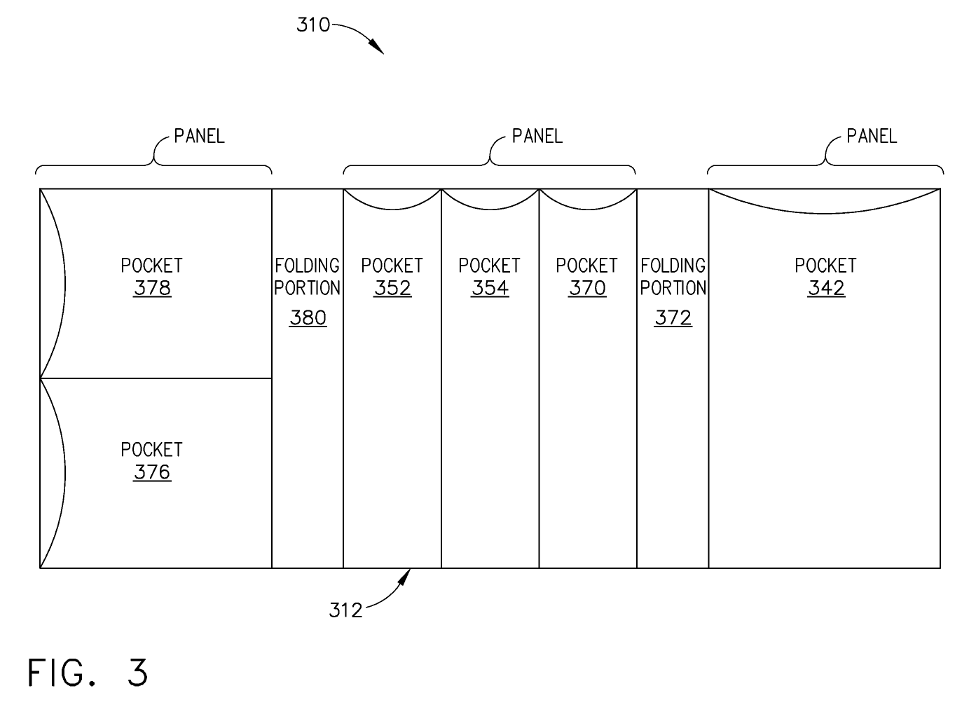

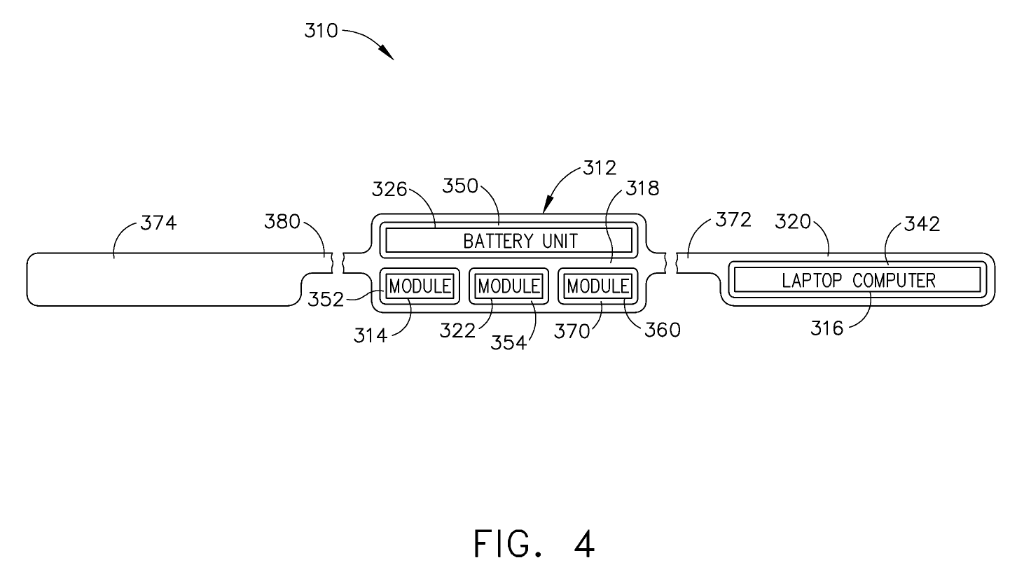

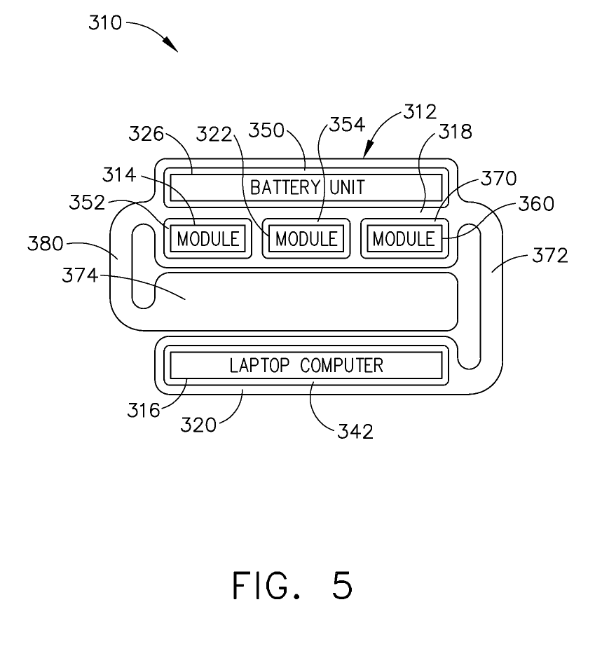

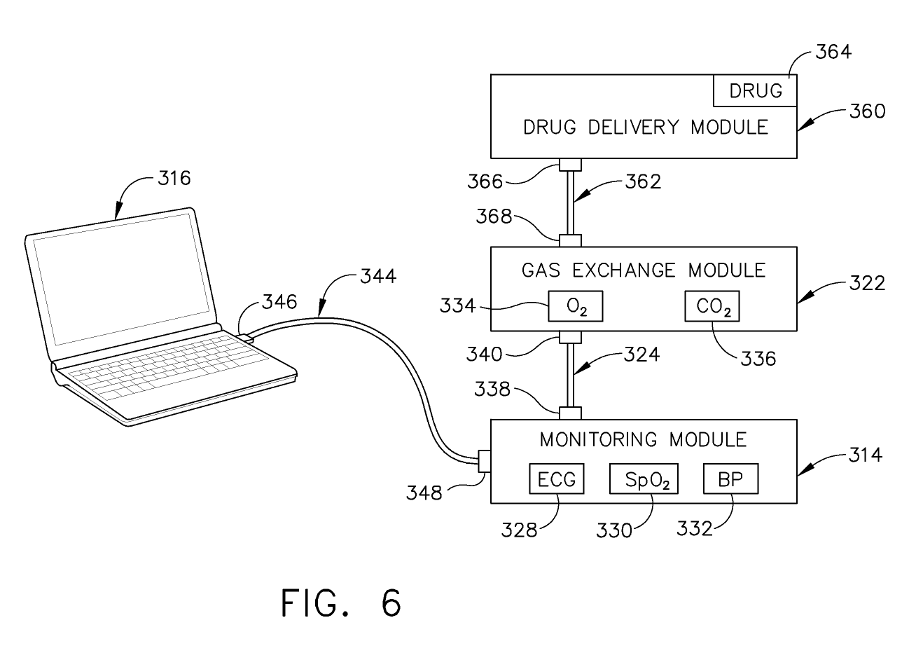

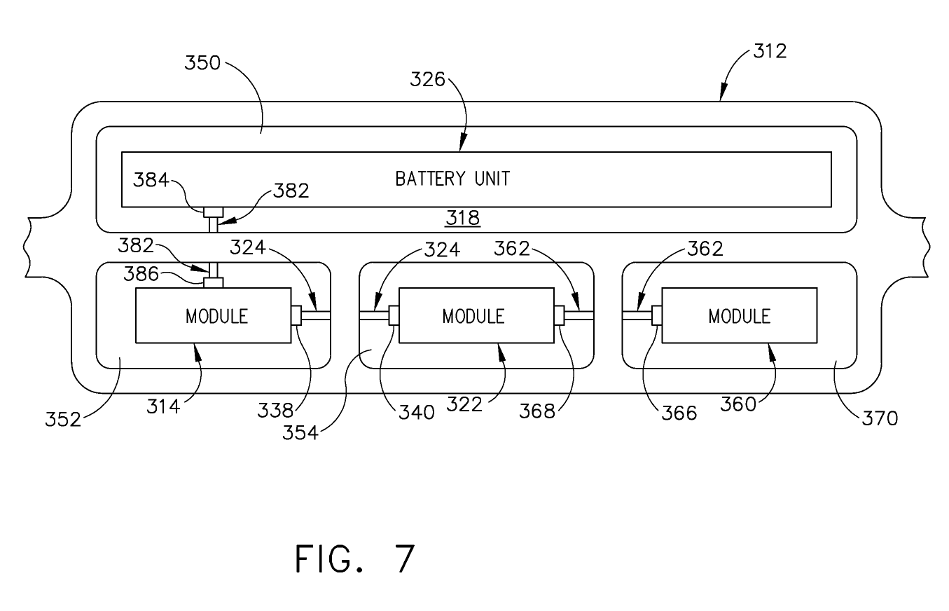

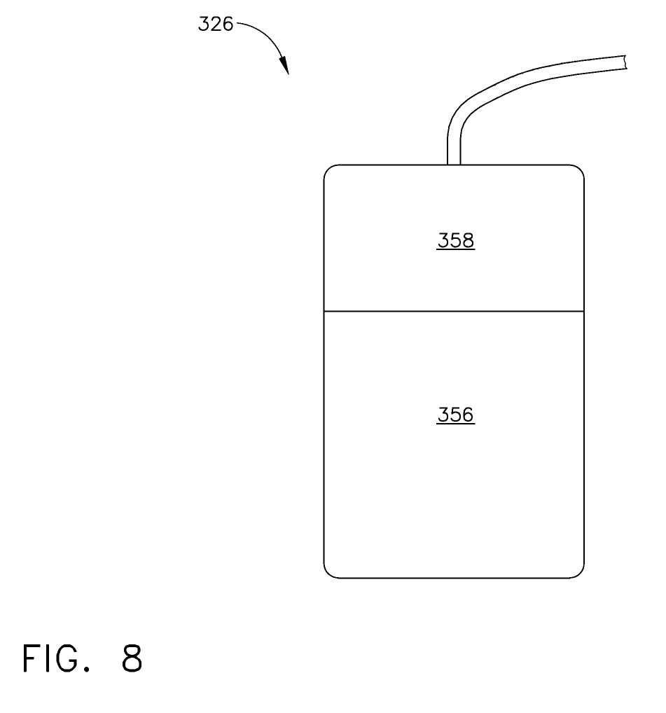

A third embodiment of the invention is shown in FIGS. 3-8. A first expression of the embodiment of FIGS. 3-8 is for a medical system 310 including a folding backpack pouch 312, a no-tool-user-replaceable patient monitoring module 314, and a no-tool-user-removable laptop computer 316. The backpack pouch 312 is adapted to be carried by a person and includes first and second panels 318 and 320 together having an open position (see FIGS. 3-4) wherein the first and second panels 318 and 320 are disposed side by side and having a closed position (see FIG. 5) wherein one of the first and second panels 318 and 320 overlies the other of the first and second panels 318 and 320. The patient monitoring module 314 is held by the first panel 318 and is adapted to measure at least one physiological parameter of a patient. The laptop computer 316 is held by the second panel 320, wherein the laptop computer 316 is operatively connectable to the patient monitoring module 314 and is adapted to display the measured at-least-one physiological parameter of the patient.

It is noted that a backpack pouch is a pouch which can be carried by a person, either by being adapted with carrying straps or (as in the example of FIG. 5) by being adapted to be placed in a knapsack having carrying straps, allowing use of the medical system in the field. It is also noted that each of the first and second panels 318 and 320 may be rigid or flexible.

In one implementation of the first expression of the embodiment of FIGS. 3-8, the medical system 310 also includes a no-tool-user-replaceable patient gas-exchange module 322 held by the first panel 318 and adapted to exchange at least one gas with the patient, wherein the laptop computer 316 is operatively connectable to the patient gas-exchange module 322.

A second expression of the embodiment of FIGS. 3-8 is for a medical system 310 including a folding backpack pouch 312, a no-tool-user-replaceable patient monitoring module 314, a no-tool-user-replaceable patient gas-exchange module 322, a first inter-module connecting cable 324 (e.g., a ribbon cable), a battery unit 326, and a no-tool-user-removable laptop computer 316. The backpack pouch 312 is adapted to be carried by a person and includes first and second panels 318 and 320 together having an open position (see FIGS. 3-4) wherein the first and second panels 318 and 320 are disposed side by side and having a closed position (see FIG. 5) wherein one of the first and second panels 318 and 320 overlies the other of the first and second panels 318 and 320.

The patient monitoring module 314 is held by the first panel 318 and includes an ECG (electrocardiogram) medical unit 328, an SpO₂ (saturation of peripheral oxygen) medical unit 330, and a blood pressure (BP) medical unit 332 each operatively connectable to a patient. The patient gas-exchange module 322 is held by the first panel 318 and includes an oxygen-delivery (O₂-delivery) medical unit 334 and a CO₂ (carbon dioxide) monitoring medical unit 336 each operatively connectable to the patient. The first inter-module connecting cable 324 is affixed to the backpack pouch 312, has a plug 338 connected to the patient monitoring module 314, and has a plug 340 connected to the patient gas-exchange module 322. The battery unit 326 is held by the first panel 318 and is adapted to power the patient monitoring module 314 and the patient gas-exchange module 322.

The laptop computer 316 is held by the second panel 320. The laptop computer 316 is operatively connectable to the patient monitoring module 314 and is adapted to display data from the ECG, SpO₂, and blood pressure medical units 328, 330, and 332, wherein the laptop computer 316 is operatively connectable to the patient gas-exchange module 322 through the patient monitoring module 314. The laptop computer 316 is adapted to display data from the CO₂ monitoring medical unit 336 and to control the oxygen-delivery medical unit 334 to deliver oxygen to the patient. It is noted that the term “oxygen” includes oxygen-enriched air, that “oxygen” is considered to be a drug, and that an oxygen-delivery medical unit 334 is a medical unit capable of delivering oxygen. It is also noted that other gases (such as nitrous oxide) can be used instead of oxygen in the oxygen-delivery medical unit 334 for a particular medical procedure. It is further noted that an example of an SpO₂ medical unit 330 is a pulse oximeter, and an example of a CO₂ monitoring medical unit 336 is a capnometer.

In one implementation of the second expression of the embodiment of FIGS. 3-8, the second panel 320 includes a pocket 342 for holding the laptop computer 316, and the medical system 310 also includes a computer cable 344 (e.g., a USB or Ethernet cable) having a plug 346 connectable to the laptop computer 316 and having a plug 348 connectable to the patient monitoring module 314. In one utilization, the patient-monitoring module 314 and the patient gas-exchange module 322 remain stored in the backpack pouch 312 during medical use, and the laptop computer 316 is removed from the backpack pouch 312 during medical use. In a different implementation, not shown, a hook and loop type attachment (such as a Velcro attachment) holds the patient-monitoring module and the patient gas-exchange module to the first panel and holds the laptop computer to the second panel, wherein the laptop computer remains attached to the second panel during medical use.

In the same or a different implementation, the first panel 318 includes a pocket 350 on a back side of the first panel 318 for holding the battery unit 326. The first panel 318 includes a pocket 352 on a front side of the first panel 318 for holding the patient monitoring module 314. The first panel 318 includes a pocket 354 on the front side of the first panel 318 for holding the patient gas-exchange module 322. In one variation, the battery unit 326 includes a rechargeable battery 356 and a hand-crank battery recharger 358.

In a first enablement of the second expression of the embodiment of FIGS. 3-8, the medical system 310 includes a patient drug delivery module 360 (e.g., an IV pump assembly) and a second inter-module connecting cable 362 (e.g., a ribbon cable). The patient drug delivery module 360 is held by the first panel 318 and is operatively connectable to a patient to deliver a drug 364 to the patient. The second inter-module connecting cable 362 is affixed to the backpack pouch 312, has a plug 366 connected to the patient drug delivery module 360, and has a plug 368 connected to the patient gas-exchange module 322. The battery unit 326 is adapted to power the patient drug delivery module 360. The laptop computer 316 is operatively connectable to the patient drug delivery module 360 through the patient gas exchange module 322 and the patient monitoring module 314. The laptop computer 316 is adapted to control the patient drug delivery module 360 to control a flow rate of the drug 364 to the patient for a predetermined medical procedure using the data from at least a plurality of the ECG, SpO₂, blood pressure, and CO₂ monitoring medical units 328, 330, 332, and 336.

In one application of the first enablement, the first panel 318 includes a pocket 370 on the front side of the first panel 318 for holding the patient drug delivery module 360. In the same or a different application, the drug 364 has at least one medical effect on the patient chosen from the group consisting of a sedative effect and an analgesic effect.

In one construction of the second expression of the embodiment of FIGS. 3-8, the backpack pouch 312 includes a first folding portion 372 interconnecting the first and second panels 318 and 320 to facilitate folding. In one extension, the backpack pouch 312 includes a third panel 374 having a pocket 376 for storing multiple-patient-use items (not shown) such as an ECG cable and leads, an SpO₂ cable and probe, a blood pressure cable and cuff, and, if required, an oxygen bottle adapted to attach to the oxygen-delivery medical unit 334. In the same or a different extension, the third panel 374 has a pocket 378 for storing single-patient-use items (not shown) such as an oral/nasal cannula, and an IV line. In one variation, the backpack pouch 312 includes a second folding portion 380 interconnecting the first and third panels 318 and 374 to facilitate folding. In one modification, the medical system 310 includes a battery connection cable 382 affixed to the backpack pouch 312, having a plug 384 connected to the battery unit 326 and having a plug 386 connected to the patient-monitoring module 314. In one construction, the backpack pouch 312, including the panels, folding portions, and the pockets, is made from canvas material. Other constructions are left to the artisan.

Several benefits and advantages are obtained from one or more of the expressions of the embodiments of the invention. In one example of the first embodiment, a medical-system controller is provided which is connectable to a distributed plurality of predetermined patient monitors and which adapts its choice of patient monitors to use depending on the particular medical procedure to be performed. In one example of the second embodiment, a medical-system controller is provided which is connectable to a distributed plurality of patient monitors and a distributed multiplicity of drug-delivering medical effectors, which identifies connected one of the medical effectors and patient monitors, and which controls at least one of the connected ones of the medical effectors using at least some of the connected ones of the patient monitors for a predetermined medical procedure. In one example of the third embodiment, a medical system is provided which includes patient modules stored in a folding backpack pouch which can be carried by a person, either by being adapted with carrying straps or by being placed in a knapsack having carrying straps, allowing use of the medical system in the field.

While the present invention has been illustrated by expressions of several embodiments, and enablements, implementations, etc. thereof, it is not the intention of the applicants to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A medical system comprising a medical-system controller,
wherein the controller is operatively connectable to a distributed plurality of predetermined patient monitors to receive at least one physiological parameter measured by each of the plurality of patient monitors,
wherein the controller is adapted to choose a first group of the patient monitors for a predetermined first medical procedure and a predetermined drug-delivering first medical effector,
wherein the controller is operatively connectable to the first medical effector and, when operatively connected, is adapted to control a flow rate of a drug from the first medical effector to a patient for the first medical procedure using at least the received measured physiological parameters from the chosen first group of the patient monitors, when the controller is operatively connected to the patient monitors, without using any other physiological parameter of the patient,
wherein the controller is adapted to choose a second group of the patient monitors different from the first group for a different predetermined second medical procedure and the first medical effector; and
wherein the controller, when operatively connected to the first medical effector, is adapted to control a flow rate of a drug from the first medical effector to the patient for the second medical procedure using at least the received measured physiological parameters from the chosen second group of the patient monitors, when the controller is operatively connected to the patient monitors, without using any other physiological parameter of the patient.

2. The medical system of claim 1, wherein the controller is adapted to control the flow rate for the first medical procedure despite failure of at least one of the patient monitors of the first group of the patient monitors.

3. The medical system of claim 1, also including the plurality of the patient monitors and the first medical effector wherein each is operatively connected to the controller.

4. The medical system of claim 1,
wherein the controller is adapted to choose a third group of the patient monitors for a third medical procedure and a predetermined drug-delivering second medical effector,
wherein the controller is operatively connectable to the second medical effector and, when operatively connected, is adapted to control a flow rate of a drug from the second medical effector to the patient for the third medical procedure using at least the received measured physiological parameters from the chosen third group of the patient monitors, when the controller is operatively connected to the patient monitors, without using any other physiological parameter of the patient.

5. The medical system of claim 4, wherein the controller is adapted to control the flow rate of the drug of the second medical effector for the third medical procedure despite failure of at least one of the patient monitors of the third group of monitors.

6. The medical system of claim 4, also including the patient monitors, the first medical effector, and the second medical effector wherein each is operatively connected to the controller.

7. The medical system of claim 1,
wherein the controller is adapted to choose a fourth group of the patient monitors for the first medical procedure and a predetermined drug-delivering second medical effector,
wherein the controller is operatively connectable to the second medical effector and, when operatively connected, is adapted to control a flow rate of a drug from the second medical effector to the patient for the first medical procedure using at least the received measured physiological parameters from the chosen fourth group of the patient monitors, when the controller is operatively connected to the patient monitors, without using any other physiological parameter of the patient.

8. The medical system of claim 7, wherein the controller is adapted to control the flow rate of the drug of the first medical effector for the first medical procedure despite failure of the second medical effector.

9. A medical system comprising a medical-system controller,
wherein the controller is connectable to up to a distributed plurality P of patient monitors which are operatively connectable to a patient to each measure at least one physiological parameter of the patient and which are adapted to identify themselves when queried,
wherein the controller is connectable to up to a distributed multiplicity M of drug-delivering medical effectors which are operatively connectable to the patient and which are adapted to identify themselves when queried,
wherein the controller is adapted to identify, by querying, connected ones of the M medical effectors and connected ones of the P patient monitors, and
wherein the controller is adapted to control at least one of the identified connected ones of the M medical effectors using at least the physiological parameters supplied by at least some of the identified connected ones of the P patient monitors for a predetermined medical procedure.

10. The medical system of claim 9, also including the plurality P of the patient monitors and the multiplicity M of the medical effectors.
